(19) United States
(12) Patent Application Publication
(10) Pub. No.: US 2003/0019005 A1
(43) Pub. Date: Jan. 30, 2003

(54) Direct vision enhancer

(76) Inventor: Peggy Allene Burnett, Hickory Ridge, AR (US)

Correspondence Address: Peggy A. Burnett, 112 CR 111, Hickory Ridge, AR, 72347, US

(21) Appl. No.: 09/916,461

(22) Filed: Jul. 27, 2001

(51) Int. Cl. A61F 9/00

(52) U.S. Cl. 2/15; 2/10

(57) ABSTRACT

An apparatus worn across the forehead and above the ears, such as a sun visor or cap is worn, and extends downward to the cheek bone which has the purpose of enhancing direct vision by diminishing peripheral vision. A devise that consists of a bill or visor (10) that flows into an area of continuous-contouring bill or visor (12) that cups and continues downward to form a peripheral vision diminisher (14) then flows upward into an area of continuous-contouring peripheral vision diminisher into leg or strap (16) which joins the leg or strap (18). The apparatus is secured to the head by use of a band (20) or leg (18). Possible variations include, but are not limited to, lighting in the bill or visor (22) and a pair that attaches to eye wear (23). I contend that direct vision and peripheral vision are two separate sight senses that sometime work in conflict in some people, particularly children, to such a degree that peripheral vision dominance prevents optimal learning from taking place. I further state that an appealing, comfortable direct vision enhancer which diminishes peripheral vision is strongly needed. A direct vision enhancer will improve or increase learning, attention span, behaviors, self control, comprehension, train of thought, successfully completing tasks, sports scores, etc. for people of all ages. The direct vision enhancer simulates the universal practice of cupping ones hands around ones face to block or diminish distractions thereby enhancing direct vision.

### CROSS-REFERENCE TO RELATED APPLICATIONS

Not applicable.

### FEDERALLY SPONSORED RESEARCH OR DEVELOPMENT

This application is the sole ownership and development of Peggy A. Burnett and has received no assistance from Federally sponsored research or development.

### BACKGROUND OF THE INVENTION

This invention relates to enhancing direct vision by diminishing peripheral vision. The bill of a sun visor or cap extends horizontally, cups downward close to the cheek bone, contours back to the leg or strap near the ear, and has the purpose of enhancing direct vision by deminishing peripheral vision. Humans have two fields of vision: direct vision—that which is before us and peripheral vision—that which is beside us. All forms of animals often need blinders which block peripheral distractions and also enhance direct vision. Man attached blinders to the horses' bridles in order for horses to run straight. Eagles have the ability to completely self block and focus more acutely. But, humans need an aid to enhance direct vision. Inventors have created several types of blinders. U.S. Pat. No. 5,007,110 to Gilbert (1991) discloses blinders which fold up under the bill of a cap or visor. The awkward, squared structure and appearance deter from the comfort and usability of the product. Also, having more parts adds to the expense of the product. U.S. Pat. No. 4,298,991 to Recenello (1980) discloses attachable peripheral view blinders to existing eyeglasses or to eyeglass like frames. The structure of the two attachable blinders are awkward and too troublesome to attach. U.S. Pat. No. 5,661,534 to Gill (1996) discloses a peripheral vision limiting visor, a structure that has the appearance of being square. FIG. 7 which is described as “concave” has an appearance of goggles which rest on the nose.

Blinders have been proposed by other inventors, but their structures have been squared with numerous parts which makes their use awkward and uncomfortable. Blinders have been attached to caps such as U.S. Pat. No. 5,901,371 to Lee (1999) and to fishing hats such as U.S. Pat. No. 5,884,334 to Collette (1999).

### SUMMARY

In accordance with the present invention a Direct Vision Enhancer comprises a bill or visor which cups and continuously-contours downward to the cheek bone creating a peripheral vision diminisher which continuously-contours back to the leg or strap near the ear. My invention simulates cupping your hands around your face whenever you need to focus attention on a task before you.

Objects and Advantages

Accordingly, a direct vision enhancer has many objects and advantages:

(a) to enhance direct vision

(b) to diminish peripheral vision distractions

(c) to increase the ability to pay better attention to whomever or whatever is before you; such as, teacher, book, writing, drawing, movie, sports, any task, etc.

(d) to increase attention span

(e) to increase abilities to learn more

(f) to improve test scores because wearer can pay attention to the task before him

(g) to be able to focus without cupping your hands to your face

(h) to improve classroom behaviors due to less distractions

(i) to be able to practice better self-control

(j)to focus on work before you in the work place such as office or computer lab

(k) to diminish the blinding sun's glare when driving, playing sports, fishing, etc.

(l) to shade the eyes

Further objects and advantages will be revealed as people realize the need for this invention and begin wearing them comfortably.

### DRAWING FIGURES

In the drawings, closely related figures have the same number.

FIG. 1 is a basic direct vision enhancer whereby a small bill contours downward to form a peripheral vision diminisher then back up to the leg. FIG. 1A shows a side version of how the invention will look while being worn.

FIG. 2 is a direct vision enhancer with a visor size bill which contours downward to form a peripheral vision diminisher then back up to the leg.

FIG. 3 is a direct vision enhancer with a visor which contours downward to form a peripheral vision diminisher then back up to a strap which holds it onto the head.

FIG. 4 is a cap whose bill or visor contours downward to form a peripheral vision diminisher then contours upward to the base of the cap. FIG. 4 also shows that the invention can be hinged at the top of the peripheral vision diminisher or other locations in order to fold when not in use.

FIG. 5 shows that solar or battery operated lights can go under the bill or visor.

FIG. 6 shows a contoured peripheral vision diminisher which can attach to eye wear with bands or clip-ons

FIG. 7 shows that caps or visors constructed from materials other than molded plastics would need darts on each side of the bill and darts on the peripheral vision diminisher that attaches to the bill in order to achieve the needed cupped effect.

### REFERENCE NUMERALS IN DRAWINGS

10 bill or visor

12 area of continuous-contouring bill into peripheral vision diminisher

14 peripheral vision diminisher

16 area of continuous-contouring peripheral vision diminisher into leg or strap

18 leg or strap

20 band

22 lights

23 attachments to eye wear

24 darts in bill

25 darts in peripheral vision enhancer

### DESCRIPTION

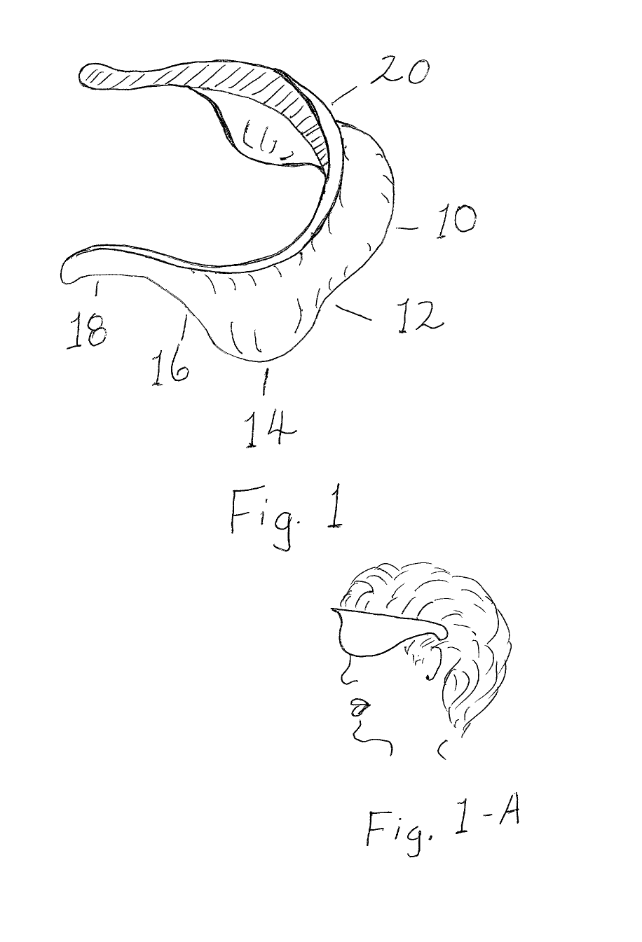

FIGS. 1-6—Preferred Embodiment

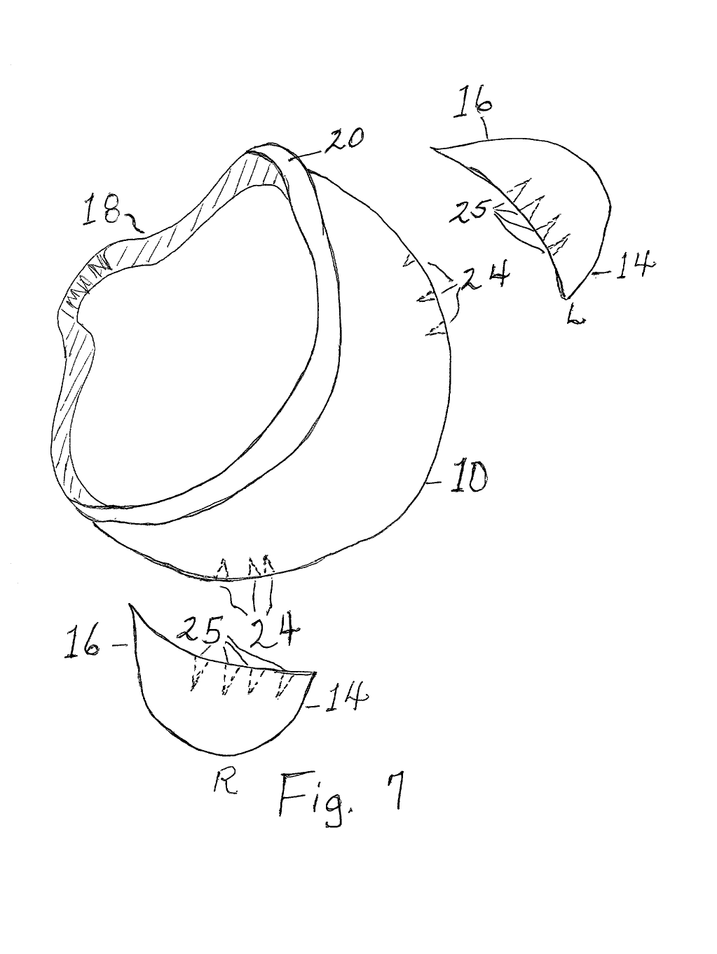

Reference numerals 10 through 20 are interchangeable in that they represent the same location in FIGS. 1-7 except that FIG. 6 does not have a bill. In FIG. 1 the depth of the bill will be from about 2 cm for a small size to about 4 cm for a large size. The continuous-contouring area from the bill 12 flows downward into about 3 cm for the small size to about 6 cm for the large size peripheral vision diminisher 14. The peripheral vision diminisher then flows upward into the continuous-contouring area 16 into the leg or strap 18. The apparatus is secured to the forehead by the band 20. The frontal shape comprising 12 and 14 is cupped or curved inward slightly toward the face to simulate the universal practice of cupping ones hands around ones face to block or diminish peripheral distractions. The invention is worn similar to a sun visor whereby it fits on the forehead and above the ears. FIG. 1 with the small bill is primarily for indoor use in the home, school, library, workplace, computer laboratory, indoor sports, etc. The color for the interior should not be distracting and will be medium to dark, preferably medium gray to light black. According to an ophthalmologists, black is a threatening color and medium to dark gray is the best color. The exterior can be any color or combination of colors. The most desired material for making FIG. 1 is plastic, such as polystyrene. This material is readily available in color, reasonably priced, and easily formed by a wide range of plastic processors, such as injection molding. The molded plastic apparatus can be uncovered or covered with cloth such as twill or terry cloth. FIG. 1 should be opaque and is the simplest and most inexpensive to make. FIG. 1-A shows what the direct vision enhancer will look like when worn.

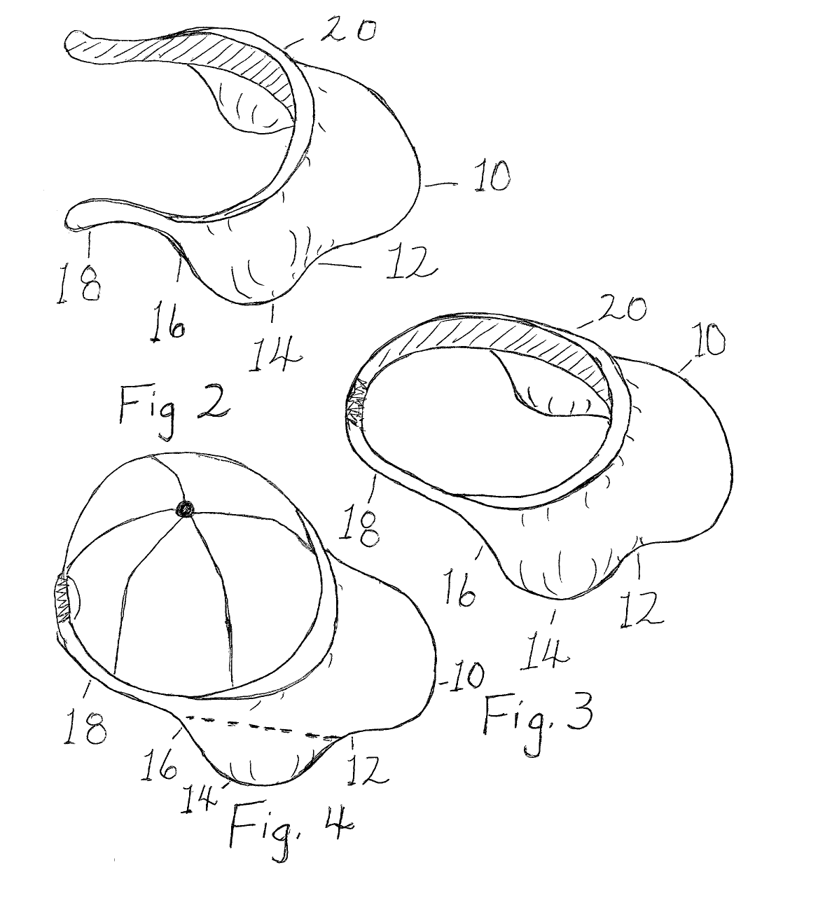

The descriptions for FIGS. 2, 3, and 4 are virtually the same as for FIG. 1; therefore, only the differences will be noted. FIGS. 2, 3, and 4 show the deeper bill or visor which will range from about 4 cm to about 7 cm depending on size and are primarily for outdoor use. The deeper bills or visors do not need to incurve but can slightly. FIGS. 2, 3, and 4 can be made from either opaque or tinted-transparent material or a combination thereof. The direct vision enhancer constructed from a tinted-transparent material with ultraviolet rays protection, such as the material for making sunglasses lenses, or a combination of opaque and transparent, will be a much needed apparatus when playing golf, bowling, chess, fishing, hunting, driving, etc. Its purpose, to enhance direct vision by diminishing peripheral vision, will be accomplished plus it will provide the needed shade for the face while outdoors. A sweatband can be attached inside the band. When made from an opaque material, more peripheral vision will be diminished and the color will be the same as for FIG. 1. The apparatus can be constructed by using a plastic mold or material with a stiffener for needed areas. Transparent material of ultraviolet rays protection will the most desirable material for outdoor use, and also will be the most expensive. The deeper bills or visors in FIGS. 2, 3, and 4 do not need to curve inward but can slightly.

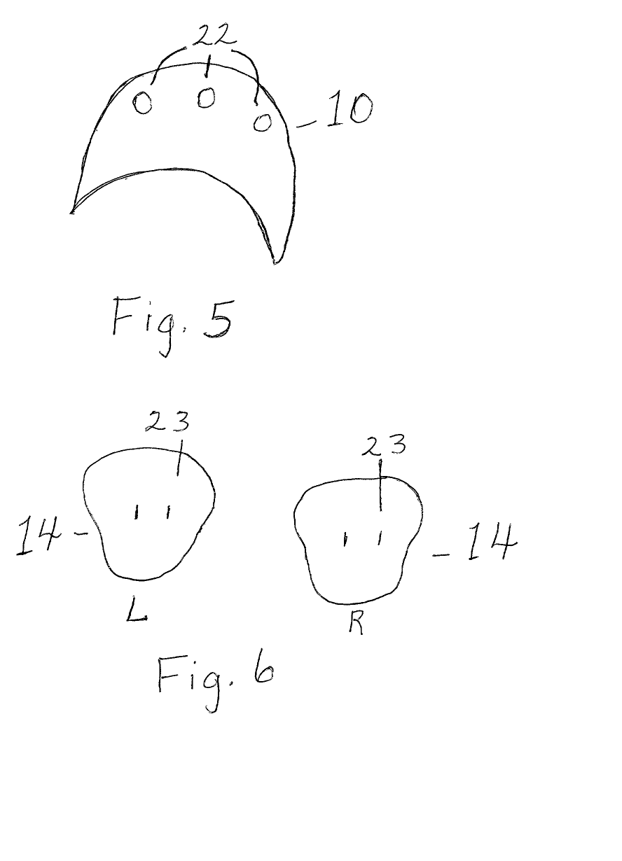

FIG. 5 is a possible added lighting aid for indoors or outdoors.

FIG. 6 is a pair of direct vision enhancers without the bill should the need arise.

The overall description for FIG. 7 is the same as FIGS. 1-5. FIG. 7 illustrates how materials other than molded plastic will need darts, or folded slits, in the bill and the peripheral vision diminisher in order to accomplish the cupped effect.

Plastic contoured molding will be the preferred basic means for construction for FIGS. 1-7. Materials such as terry cloth and/or twill to cover the molded plastic will aid in comfort and appearance.

Alternative Embodiments

The width of a bill or visor can vary if the demand justifies. The width can be narrow enough so that the peripheral vision diminisher can fit close to the face or the width can be greater to accommodate eye wear. It is hoped that a mutual bill and/or visor width will suffice both. One possible extension is that the area going into the leg can extend and protrude enough to shade the tops of wearer's ears. Should demand justify, a means for folding the invention more compactly will be devised. Also, use of different colors or shades for tinting may prove to be beneficial for various situations.

Advantages

From the description above, a number of advantages of my direct vision enhancer become evident:

(a) students will make better grades and higher test scores

(b) classrooms will have less disruptions due to inappropriate behaviors of students

(c) less students will be labeled as at-risk students, slow learners, attention deficit disorder, oppositional defiant disorder, trouble makers, etc.

(d) people's comprehension will increase

(e) students will be able to listen and tune-in to the teacher more acutely

(f) the sense of direct vision will be enhanced

(g) learning will be easier

(h) sports scores will improve—less distractions for golfers, bowlers, tennis players, chess, checkers, fishermen, hunters, etc.

(i) people will have more eye protection

(j) people will not always need to wear sunglasses when outdoors

(k) self-concept will improve

(l) tasks will be completed more accurately in less time

(m) possibility that dyslexics can be helped

This invention is not a cure-all, but it is an aid that I invented when trying to address the problem of “why some students have difficulty learning”. Even though “blinders” are not new, my invention of direct vision enhancer by diminishing peripheral vision with its objects, advantages, and purposes has numerous possibilities for even more uses, and the continuous-contoured design will be universally accepted.

Operation

FIG. 1 shows a perspective view of a basic version of my direct vision enhancer. Various size bills or visors 10 serve as a connector which continuously-contours 12 and cups downward to form a peripheral vision diminisher 14 then continuously-contours 16 upward into the leg or strap 18. The apparatus is secured to the head by a band 20.

Theory of Operation

While trying to find ways to help students comprehend more of the task before them, I remembered how when one sense is decreased, another sense is increased. So, I contemplated ways to help students pay closer attention to their teacher or successfully complete the task before them. Even though the sense of sight has traditionally been thought of as one sense, I contend that direct vision and peripheral vision are two separate senses of sight. Scientifically, we do know that people can be legally blind and still have peripheral vision. So, logically it appears that direct vision and peripheral vision are actually two separate senses that normally work in conjunction but in some people, particularly some children, work in a conflicting, abnormal manner. Whenever this abnormality occurs, people, particularly children, develop peripheral vision dominance to such a degree that they cannot attend to the task before them because they constantly are distracted by what is in their peripheral vision. These distractions prevent people from focusing, or optimally comprehending from whomever or whatever is in their direct vision. Some students are at-risk students or slow learners and others have Attention Deficit Disorder or Oppositional Defiant Disorder, etc. I further contend that these problems are hampered by peripheral vision distractions to such a degree that peripheral vision dominance is one cause of these problems. Dyslexia might also be helped by using a combination of tinted-transparent, such as yellow, bill and opaque peripheral vision diminisher. I am not advocating that peripheral vision dominance is the only cause for these problems, but I do contend that it compounds these problems. I further contend that this invention will have a positive impact on these problems.

I then developed several structures for a direct vision enhancer until I came up with my present invention. This invention simulates the universal practice of cupping your hands around your face to block out distractions. I, and everyone else that I discussed this invention with, use this practice. Then I began to expound on the many uses as mentioned in objects and advantages.

Conclusion, Ramifications, and Scope of Invention

Thus the reader will see that the direct vision enhancer provides an effective, efficient, economical device that can be used by people of almost any age. The simplicity of the structure, patterned after cupping your hands around your face, and the smooth continuous-contoured flow creates a needed product. Educators, especially Special Education teachers, have told me that they want one for every child in their classroom. The appealing appearance of the smooth contouring design will help people accept and embrace this new invention. The direct vision enhancer fits in the middle of the forehead like a sun visor and probably can be worn along with eye wear. I do not claim that golfers, bowlers, or other sports enthusiasts will improve their abilities, but diminishing peripheral distractions will help with concentration on whatever is before them, and that should increase their scores. I further know that IQ's will not increase, but IQ scores probably will. With direct vision enhanced and concentration increased, one can complete each task faster with more accuracy. I think direct vision enhancers will become a needed educational aid as well as a recreational aid. I further think that the tinted-transparent models should be worn while driving since peripheral vision often prevents wrecks. As I was driving North recently, I realized that my left hand was cupped to the left side of my face blocking the glaring sun, and I know that wearing the direct vision enhancer, constructed from tinted transparent ultraviolet rays protection material, will diminish the blinding sun rays and protect eyes.

While my above description contains many specificities, these should not be construed as limitations on the scope of the invention. Other variations may become apparent as the invention becomes widely accepted such as adjustments within the invention, means to compact or fold for storage, various materials to use, etc. Accordingly, the scope of the invention should be determined not by the embodiment(s) illustrated, but by the appended claims and their legal equivalents.

I claim:

1. An apparatus for enhancing direct vision comprising:
(a) a horizontally extended bill or visor which curves downward creating an area of continuous-contouring bill which cups and continues downward to form a peripheral vision diminisher on each side of the wearer's head;
(b) said peripheral vision diminisher extends beyond wearer's eyes, is of a depth to wearer's cheekbone, curves toward the ear, and continuously-contours in an upward flow to the leg or strap of the head gear
(c) said area of contouring bill or visor and said frontal portion of the peripheral vision diminisher incurve slightly to simulate cupping ones hands around ones face to slightly or substantially diminish peripheral vision, depending on purpose and material, whereby enhancing direct vision.
